(19) United States
(12) Patent Application Publication
(10) Pub. No.: US 2009/0076349 A1
(43) Pub. Date: Mar. 19, 2009

(54) Adherent Multi-Sensor Device with Implantable Device Communication Capabilities

(75) Inventors: Imad Libbus, Saint Paul, MN (US); Yatheendhar D. Manicka, Woodbury, MN (US); Scott Mazar, Woodbury, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corventis, Inc., San Jose, CA (US)

(21) Appl. No.: 12/209,508

(22) Filed: Sep. 12, 2008

Related U.S. Application Data
(60) Provisional application No. 61/055,666, filed on May 23, 2008.
(60) Provisional application No. 60/972,537, filed on Sep. 14, 2007.
(60) Provisional application No. 60/972,316, filed on Sep. 14, 2007.
(60) Provisional application No. 60/972,333, filed on Sep. 14, 2007.
(60) Provisional application No. 60/972,336, filed on Sep. 14, 2007.

(51) Int. Cl. A61B 5/02

(52) U.S. Cl. 600/301

(57) ABSTRACT

A system for physiological monitoring and prediction. The system includes a detecting system, one or more medical devices and a remote monitoring system. The detecting system includes an adherent device configured to be coupled to a patient, the adherent device including a plurality of sensors that provide an indication of at least one physiological event of a patient and a wireless communication device coupled to the plurality of sensors and configured to transfer patient data from the plurality of sensors and one or more medical treatment devices to the remote monitoring system. The one or more medical treatment devices are coupled to the wireless communication device to communicate wirelessly with the plurality of sensors, with at least one medical treatment device being an implanted device. The remote monitoring system is coupled to the wireless communication device, wherein the remote monitoring system uses data from the sensors and the one or more medical treatment devices to determine heart failure status and predict impending decompensation of the patient.

### CROSS-REFERENCES TO RELATED APPLICATIONS

The present application claims the benefit under 35 USC 119(e) of U.S. Provisional Application Nos. 60/972,316, 60/972,333, 60/972,336 and 60/972,537, all filed Sep. 14, 2007, and 61/055,666 filed May 23, 2008; the full disclosures of which are incorporated herein by reference in their entirety.

The subject matter of the present application is related to the following applications: 60/972,512; 60/972,329; 60/972,354; 60/972,616; 60/972,363; 60/972,343; 60/972,581; 60/972,629; 60/972,359; 60/972,340 all of which were filed on Sep. 14, 2007; 61/046,196 filed Apr. 18, 2008; 61/047,875 filed Apr. 25, 2008; 61/055,645, 61/055,656, 61/055,662 all filed May 23, 2008; and 61/079,746 filed Jul. 10, 2008.

The following applications are being filed concurrently with the present application, on Sep. 12, 2008: Attorney Docket Nos. 026843-000110US entitled “Multi-Sensor Patient Monitor to Detect Impending Cardiac Decompensation Prediction”; 026843-000220US entitled “Adherent Device with Multiple Physiological Sensors”; 026843-000410US entitled “Injectable Device for Physiological Monitoring”; 026843-000510US entitled “Delivery System for Injectable Physiological Monitoring System”; 026843-000620US entitled “Adherent Device for Cardiac Rhythm Management”; 026843-000710US entitled “Adherent Device for Respiratory Monitoring”; 026843-000810US entitled “Adherent Athletic Monitor”; 026843-000910US entitled “Adherent Emergency Monitor”; 026843-001320US entitled “Adherent Device with Physiological Sensors”; 026843-001410US entitled “Medical Device Automatic Start-up upon Contact to Patient Tissue”; 026843-001900US entitled “System and Methods for Wireless Body Fluid Monitoring”; 026843-002010US entitled “Adherent Cardiac Monitor with Advanced Sensing Capabilities”; 026843-002410US entitled “Adherent Device for Sleep Disordered Breathing”; 026843-002710US entitled “Dynamic Pairing of Patients to Data Collection Gateways”; 026843-003110US entitled “Data Collection in a Multi-Sensor Patient Monitor”; 026843-003210US entitled “Adherent Multi-Sensor Device with Empathic Monitoring”; 026843-003310US entitled “Energy Management for Adherent Patient Monitor”; and 026843-003410US entitled “Tracking and Security for Adherent Patient Monitor.”

### BACKGROUND OF THE INVENTION

### Field of the Invention

This invention relates generally to systems and methods that use wireless physiological monitoring and prediction, and more particularly to systems and methods for heart failure patient monitoring.

Frequent monitoring of patients permits the patients' physician to detect worsening symptoms as they begin to occur, rather than waiting until a critical condition has been reached. As such, home monitoring of patients with chronic conditions is becoming increasingly popular in the health care industry for the array of benefits it has the potential to provide. Potential benefits of home monitoring are numerous and include: better tracking and management of chronic disease conditions, earlier detection of changes in the patient condition, and reduction of overall health care expenses associated with long term disease management. The home monitoring of a number of diverse “chronic diseases” is of interest, where such diseases include diabetes, dietary disorders such as anorexia and obesity, anxiety, depression, epilepsy, respiratory diseases, AIDS and other chronic viral conditions, conditions associated with the long term use of immunosuppressants, e.g. in transplant patients, asthma, chronic hypertension, chronic use of anticoagulants, and the like.

Of particular interest in the home monitoring sector of the health care industry is the remote monitoring of patients with heart failure (HF), also known as congestive heart failure. HF is a syndrome in which the heart is unable to efficiently pump blood to the vital organs. Most instances of HF occur because of a decreased myocardial capacity to contract (systolic dysfunction). However, HF can also result when an increased pressure-stroke-volume load is imposed on the heart, such as when the heart is unable to expand sufficiently during diastole to accommodate the ventricular volume, causing an increased pressure load (diasystolic dysfunction).

In either case, HF is characterized by diminished cardiac output and/or damming back of blood in the venous system. In HF, there is a shift in the cardiac function curve and an increase in blood volume caused in part by fluid retention by the kidneys. Indeed, many of the significant morphologic changes encountered in HF are distant from the heart and are produced by the hypoxic and congestive effects of the failing circulation upon other organs and tissues. One of the major symptoms of HF is edema, which has been defined as the excessive accumulation of interstitial fluid, either localized or generalized.

HF is the most common indication for hospitalization among adults over 65 years of age, and the rate of admission for this condition has increased progressively over the past two decades. It has been estimated that HF affects more than 3 million patients in the U.S. (J. B. O' Connell et al., J. Heart Lung Transpl. (1993) 13(4):S107-112).

In the conventional management of HF patents, where help is sought only in crisis, a cycle occurs where patients fail to recognize early symptoms and do not seek timely help from their care-givers, leading to emergency department admissions (Miller, P. Z., 1995, “Home monitoring for congestive heart failure patients,” Caring Magazine, August 1995: 53-54). Recently, a prospective, randomized trial of 282 patients was conducted to assess the effect of the intervention on the rate of admission, quality of life, and cost of medical care. In this study, a nurse-directed, multi disciplinary intervention (which consisted of comprehensive education of the patient and family, diet, social-service consultation and planning, review of medications, and intensive assessment of patient condition and follow-up) resulted in fewer readmissions than the conventional treatment group and a concomitant overall decrease in the cost of care (M. W. Rich et al., New Engl. J. Med. (1995) 333:1190-95).

Similarly, comprehensive discharge planning and a home follow-up program was shown to decrease the number of readmissions and total hospital charges in an elderly population (M. Naylor et al., Amer. College Physicians (1994) 120:999-1006). Therefore, home monitoring is of particular interest in the HF management segment of the health care industry.

Another area in which home-monitoring is of particular interest is in the remote monitoring of a patient parameter that provides information on the titration of a drug, particularly with drugs that have a consequential effect following administration, such as insulin, anticoagulants, ACE inhibitors, .beta.-blockers, etc.

Although a number of different home monitoring systems have been developed, there is continued interest in the development of new monitoring systems. Of particular interest would be the development of a system that provides for improved patient compliance, ease of use, etc. Of more particular interest would be the development of such a system that is particularly suited for use in the remote monitoring of patients suffering from HF.

There is a need for an improved home monitoring of patients with chronic conditions. There is a further need for an improved heart failure (HF) monitoring system.

### BRIEF SUMMARY OF THE INVENTION

In a first aspect, embodiments of the present invention provide a system for physiological monitoring and prediction. The system includes a detecting system, one or more medical treatment devices and a remote monitoring system. The detecting system includes an adherent device configured to be coupled to a patient, the adherent device including a plurality of sensors that provide an indication of at least one physiological event of a patient and a wireless communication device coupled to the plurality of sensors and configured to transfer patient data from the plurality of sensors and one or more medical treatment devices to the remote monitoring system. The one or more medical treatment devices are coupled to the wireless communication device to communicate wirelessly with the plurality of sensors, with at least one medical treatment device being an implanted device. The remote monitoring system is coupled to the wireless communication device, wherein the remote monitoring system uses data from the sensors and the one or more medical treatment devices to determine heart failure status and predict impending decompensation of the patient.

In many embodiments, the plurality of sensors serve as a communication hub for the one or more medical devices.

In many embodiments, may be a bioimpedance sensor, a heart rate sensor, a heart rhythm sensor, a HRV sensor, HRT sensor, a heart sounds sensor, a respiration rate sensor, a respiration rate variability sensor, a respiratory sounds sensor, a SpO₂ sensor, a blood pressure sensor, an activity sensor, a posture sensor, a wake/sleep sensor, an orthopnea sensor, a temperature sensor, a heat flux sensor and an accelerometer.

In many embodiments, the medical treatment device is a cardiac rhythm management device.

In many embodiments, the communication includes coordinating sensor data and therapy delivery while transmitting and receiving data from the remote monitoring system.

In many embodiments, the plurality of sensors is configured to communicate with external systems. The external systems may be a weight scale, blood pressure cuff, cardiac rhythm management device, a medical treatment device and a medicament dispenser

In many embodiments, the medical treatment device is a cardiac rhythm management device.

In many embodiments, the communication includes coordinating sensor data and therapy delivery while transmitting and receiving data from the remote monitoring system.

In many embodiments, the plurality of sensors is configured to communicate with external systems. The external systems may include a weight scale, blood pressure cuff, cardiac rhythm management device, a medical treatment device and a medicament dispenser.

In many embodiments, the plurality of sensors is configured to coordinate data sharing between external systems allowing for sensor integration across the external systems.

In many embodiments, the plurality of sensors is configured to coordinate data sharing between devices allowing for sensor integration across the devices. The devices may include a weight scale, blood pressure cuff, cardiac rhythm management device, a medical treatment device and a medicament dispenser. The coordination of sensors of the plurality of sensors provides for new pacing, sensing, and defibrillation vectors.

In many embodiments, the plurality of sensors is configured to switch between different modes. The modes may include a stand alone mode with communication directly with a remote monitoring system, communication directly with any one of the one or more medical devices, coordination between the one or more medical devices coupled to the plurality of sensors and different device communication protocols.

In many embodiments, the plurality of sensors is configured to deactivate selected sensors to reduce redundancy.

In another aspect, embodiments of the present invention provide an adherent multi-sensor device. The device includes a plurality of sensors that provide an indication of at least one physiological event of a patient, a processor couple to the plurality of sensors to receive data from the plurality of sensors and creates processed patient data, and a wireless communication device coupled to the processor and configured to receive patient data from one or more medical treatment devices wherein at least one medical treatment device is an implanted device, the adherent multi-sensor device serving as a communication hub for the one or more medical devices.

In many embodiments, the wireless communication device communicates data from the processor, sensors and/or the one or more medical treatment devices to a remote monitoring system configured to determine heart failure status and predict impending decompensation of the patient. The wireless communication device may be configured to receive data from the remote monitoring system and coordinate therapy delivery for the one or more medical devices.

In many embodiments, the plurality of sensors is configured to coordinate data sharing between the one or more medical devices allowing for sensor integration across the one or more medical devices.

In many embodiments, a sensor may be a bioimpedance sensor, a heart rate sensor, a heart rhythm sensor, a HRV sensor, HRT sensor, a heart sounds sensor, a respiration rate sensor, a respiration rate variability sensor, a respiratory sounds sensor, a SpO₂ sensor, a blood pressure sensor, an activity sensor, a posture sensor, a wake/sleep sensor, an orthopnea sensor, a temperature sensor, a heat flux sensor and an accelerometer.

In many embodiments, the plurality of sensors is configured to switch between different modes selected from at least one of, a stand alone mode with communication directly with a remote monitoring system, communication directly with any one of the one or more medical devices, coordination between the one or more medical devices coupled to the plurality of sensors and different device communication protocols.

In many embodiments, the plurality of sensors is configured to deactivate selected sensors to reduce redundancy.

### BRIEF DESCRIPTION OF THE DRAWINGS

FIG. 1 is a block diagram illustrating one embodiment of a patient monitoring system of the present invention.

FIGS. 2A and 2B illustrate an exploded view and side view of embodiments of an adherent device with sensors configured to be coupled to the skin of a patient for monitoring purposes;

FIG. 3 illustrates one embodiment of an energy management device that is coupled to the plurality of sensors of FIG. 1.

FIG. 4 illustrates one embodiment of present invention illustrating logic resources configured to receive data from the sensors and/or the processed patient for monitoring purposes, analysis and/or prediction purposes.

FIG. 5 illustrates an embodiment of the patient monitoring system of the present invention with a memory management device.

FIG. 6 illustrates an embodiment of the patient monitoring system of the present invention with an external device coupled to the sensors.

FIG. 7 illustrates an embodiment of the patient monitoring system of the present invention with a notification device.

FIG. 8 is a block diagram illustrating an embodiment of the present invention with sensor leads that convey signals from the sensors to a monitoring unit at the detecting system, or through a wireless communication device to a remote monitoring system.

FIG. 9 is a block diagram illustrating an embodiment of the present invention with a control unit at the detecting system and/or the remote monitoring system.

FIG. 10 is a block diagram illustrating an embodiment of the present invention where a control unit encodes patient data and transmits it to a wireless network storage unit at the remote monitoring system.

FIG. 11 is a block diagram illustrating one embodiment of an internal structure of a main data collection station at the remote monitoring system of the present invention.

FIG. 12 is a flow chart illustrating an embodiment of the present invention with operation steps performed by the system of the present invention in transmitting information to the main data collection station.

### DETAILED DESCRIPTION OF THE INVENTION

The present invention is directed to an adherent multi-sensor device capable of tracking a patient's physiological status and detecting and predicting negative physiological events. The adherent device is capable of communicating wirelessly with one or more other medical devices to share monitoring, diagnostic, and/or therapeutic capabilities.

The adherent device is designed to be affixed to the thorax of a patient and contain multiple physiological sensors for patient monitoring. The device also has wireless communication capabilities, and can communicate with an external receiver to transmit data to a remote center. In addition, the device has the capability to wirelessly communicate, via a public or proprietary communication standard, with one or more additional medical devices. An example of such a medical device is an implantable cardiac pacemaker or cardioverter defibrillator.

The adherent device may serve as a communication hub for multiple medical devices, coordinating sensor data and therapy delivery while transmitting and receiving data from a remote center. The adherent device may also communicate with additional external systems, such as cardiac rhythm management patient management systems (e.g. Boston Scientific's Latitude system, Medtronic's CareLink system, St. Jude Medical's HouseCall system). Such communication may occur directly, or via an external translator unit.

Adherent device communication can facilitate data sharing between medical devices, allowing for sensor integration across medical devices. Such coordination may also allow for new pacing, sensing, and/or defibrillation vectors.

The adherent device may switch between different modes, depending on whether or not it is communicating with additional medical devices. If communication is occurring, redundant features/sensors may be deactivated. Alternatively, the adherent device may take advantage of additional available sensor (e.g. intracardiac pressure) to enhance its monitoring and prediction capabilities.

While the present invention is intended for heart failure patient monitoring, the system may be applicable to any human application in which wireless physiological monitoring and prediction is required.

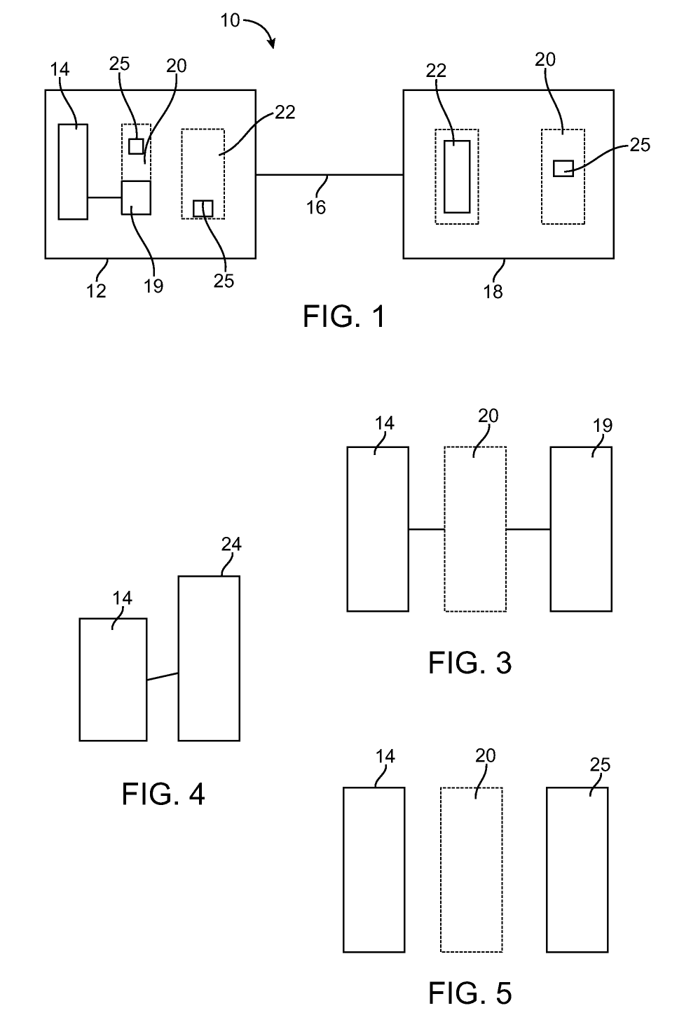

Referring to FIG. 1, one embodiment of the present invention is a patient management system, generally denoted as 10, that tracks the patient's physiological status, detects and predicts negative physiological events. In one embodiment, a plurality of sensors are used in combination to enhance detection and prediction capabilities as more fully explained below.

In one specific embodiment, the system 10 is used for decompensation prediction of a heart failure patient. A detecting system, denoted as 12, is provided. The detecting system 12 includes an adherent device that adheres to the patient's skin with a plurality of sensors 14 that provide an indication of at least one physiological event of a patient. The plurality of sensors 14 are coupled to the patient's thorax. The system 10 also includes a wireless communication device 16, coupled to the plurality of sensors 14. The wireless communication device 16 transfers patient data directly or indirectly from the plurality of sensors 14 to a remote monitoring system 18. The remote monitoring system 18 uses data from the sensors to determine heart failure status and predict impending decompensation of the patient. The detecting system 12 is activated for a first time and communicating with the remote monitoring system to register the detecting system and activate data logging. During the registration period the detecting system 12 titrates a sensitivity of each sensor 14.

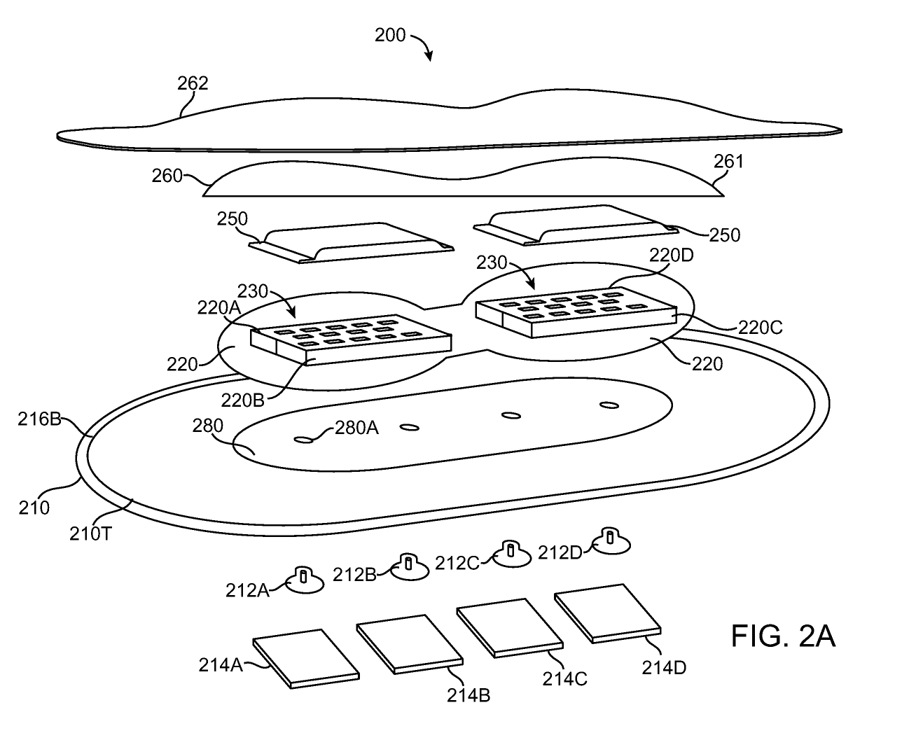

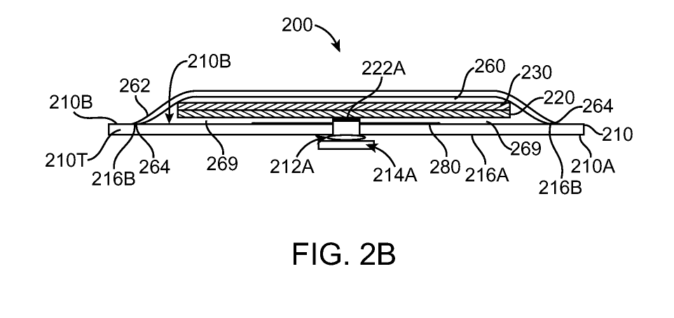

FIGS. 2A and 2B show embodiments of the plurality of sensors 14 with supported with an adherent device 200 configured to adhere to the skin. Adherent device 200 is described in U.S. application Ser. No. 60/972,537, the full disclosure of which has been previously incorporated herein by reference. As illustrated in an exploded view of the adherent device, a cover 262, batteries 250, electronics 230, including but not limited to flex circuits and the like, an adherent tape 210T, the plurality of sensors may comprise electrodes and sensor circuitry, and hydrogels which interface the plurality of sensors 14 with the skin, are provided.

Adherent device 200 comprises a support, for example adherent patch 210, configured to adhere the device to the patient. Adherent patch 210 comprises a first side, or a lower side 210A, that is oriented toward the skin of the patient when placed on the patient. In many embodiments, adherent patch 210 comprises a tape 210T which is a material, preferably breathable, with an adhesive 216A. Patient side 210A comprises adhesive 216A to adhere the patch 210 and adherent device 200 to patient P. Electrodes 212A, 212B, 212C and 212D are affixed to adherent patch 210. In many embodiments, at least four electrodes are attached to the patch, for example six electrodes. In some embodiments the patch comprises two electrodes, for example two electrodes to measure the electrocardiogram (ECG) of the patient. Gel 214A, gel 214B, gel 214C and gel 214D can each be positioned over electrodes 212A, 212B, 212C and 212D, respectively, to provide electrical conductivity between the electrodes and the skin of the patient. In many embodiments, the electrodes can be affixed to the patch 210, for example with known methods and structures such as rivets, adhesive, stitches, etc. In many embodiments, patch 210 comprises a breathable material to permit air and/or vapor to flow to and from the surface of the skin. In some embodiments, a printed circuit board (PCB), for example flex PCB 220, may be connected to upper side 200B of patch 210 with connectors. In some embodiments, additional PCB's, for example rigid PCB's 220A, 220B, 220C and 220D, can be connected to flex PCB 220. Electronic components 230 can be connected to flex PCB 220 and/or mounted thereon. In some embodiments, electronic components 230 can be mounted on the additional PCB's.

Electronic circuitry and components 230 comprise circuitry and components to take physiologic measurements, transmit data to remote center and receive commands from remote center. In many embodiments, electronics components 230 may comprise known low power circuitry, for example complementary metal oxide semiconductor (CMOS) circuitry components. Electronics components 230 comprise an activity sensor and activity circuitry, impedance circuitry and electrocardiogram circuitry, for example ECG circuitry. In some embodiments, electronics circuitry may comprise a microphone and microphone circuitry to detect an audio signal from within the patient, and the audio signal may comprise a heart sound and/or a respiratory sound, for example an S3 heart sound and a respiratory sound with rales and/or crackles. Electronics circuitry and components 230 may comprise a temperature sensor, for example a thermistor, and temperature sensor circuitry to measure a temperature of the patient, for example a temperature of a skin of the patient.

A cover 262 can extend over the batteries, electronic components and flex printed circuit board. In many embodiments, an electronics housing 260 may be disposed under cover 262 to protect the electronic components, and in some embodiments electronics housing 260 may comprise an encapsulant over the electronic components and PCB. In some embodiments, cover 262 can be adhered to adhesive patch with an adhesive. In many embodiments, electronics housing 260 may comprise a water proof material, for example a sealant adhesive such as epoxy or silicone coated over the electronics components and/or PCB. In some embodiments, electronics housing 260 may comprise metal and/or plastic. Metal or plastic may be potted with a material such as epoxy or silicone.

Cover 262 may comprise many known biocompatible cover, casing and/or housing materials, such as elastomers, for example silicone. The elastomer may be fenestrated to improve breathability. In some embodiments, cover 262 may comprise many known breathable materials, for example polyester, polyamide, and/or elastane (Spandex). The breathable fabric may be coated to make it water resistant, waterproof, and/or to aid in wicking moisture away from the patch.

Adherent device 200 comprises several layers. Gel 214A, or gel layer, is positioned on electrode 212A to provide electrical conductivity between the electrode and the skin. Electrode 212A may comprise an electrode layer. Adhesive patch 210 may comprise a layer of breathable tape 210T, for example a known breathable tape, such as tricot-knit polyester fabric. An adhesive 216A, for example a layer of acrylate pressure sensitive adhesive, can be disposed on underside 210A of patch 210. A gel cover 280, or gel cover layer, for example a polyurethane non-woven tape, can be positioned over patch 210 comprising the breathable tape. A PCB layer, for example flex PCB 220, or flex PCB layer, can be positioned over gel cover 280 with electronic components 230 connected and/or mounted to flex PCB 220, for example mounted on flex PCB so as to comprise an electronics layer disposed on the flex PCB. In many embodiments, the adherent device may comprise a segmented inner component, for example the PCB, for limited flexibility. In many embodiments, the electronics layer may be encapsulated in electronics housing 260 which may comprise a waterproof material, for example silicone or epoxy. In many embodiments, the electrodes are connected to the PCB with a flex connection, for example trace 223A of flex PCB 220, so as to provide strain relive between the electrodes 212A, 212B, 212C and 212D and the PCB. Gel cover 280 can inhibit flow of gel 214A and liquid. In many embodiments, gel cover 280 can inhibit gel 214A from seeping through breathable tape 210T to maintain gel integrity over time. Gel cover 280 can also keep external moisture from penetrating into gel 214A. Gel cover 280 may comprise at least one aperture 280A sized to receive one of the electrodes. In many embodiments, cover 262 can encase the flex PCB and/or electronics and can be adhered to at least one of the electronics, the flex PCB or the adherent patch, so as to protect the device. In some embodiments, cover 262 attaches to adhesive patch 210 with adhesive 216B. Cover 262 can comprise many known biocompatible cover, housing and/or casing materials, for example silicone. In many embodiments, cover 262 comprises an outer polymer cover to provide smooth contour without limiting flexibility. In some embodiments, cover 262 may comprise a breathable fabric. Cover 262 may comprise many known breathable fabrics, for example breathable fabrics as described above. In some embodiments, the breathable fabric may comprise polyester, polyamide, and/or elastane (Spandex™) to allow the breathable fabric to stretch with body movement. In some embodiments, the breathable tape may contain and elute a pharmaceutical agent, such as an antibiotic, anti-inflammatory or antifungal agent, when the adherent device is placed on the patient.

In one embodiment, the wireless communication device 16 is configured to receive instructional data from the remote monitoring system.

As illustrated in FIG. 3, an energy management device 19 is coupled to the plurality of sensors 14. In one embodiment, the energy management device 19 is part of the detecting system. In various embodiments, the energy management device 19 performs one or more of modulate drive levels per sensed signal of a sensor 14, modulate a clock speed to optimize energy, watch cell voltage drop-unload cell, coulomb-meter or other battery monitor, sensor dropoff at an end of life of a battery coupled to a sensor, battery end of life dropoff to transfer data, elective replacement indicator, call center notification, sensing windows by the sensors 14 based on a monitored physiological parameter and sensing rate control.

In one embodiment, the energy management device 19 is configured to manage energy by at least one of, a thermoelectric unit, kinetics, fuel cell, through solar power, a zinc air interface, Faraday generator, internal combustion, nuclear power, a micro-battery and with a rechargeable device.

The system 10 is configured to automatically detect events. The system 10 automatically detects events by at least one of, high noise states, low noise states, physiological quietness, sensor continuity and compliance. In response to a detected physiological event, patient states are identified when data collection is inappropriate. In response to a detected physiological event, patient states are identified when data collection is desirable. Patient states include, physiological quietness, rest, relaxation, agitation, movement, lack of movement and a patient's higher level of patient activity.

The system uses an intelligent combination of sensors to enhance detection and prediction capabilities, as more fully discloses in U.S. Provisional Application No. 60/972,537, filed Sep. 14, 2007, and U.S. Provisional Application No. 61/055,666, filed May 23, 2008, both entitled “Adherent Device With Multiple Physiological Sensors”, the full disclosures of which is incorporated herein by reference and as more fully explained below.

In one embodiment, the detecting system 12 communicates with the remote monitoring system 18 periodically or in response to a trigger event. The trigger event can include but is not limited to at least one of, time of day, if a memory is full, if an action is patient initiated, if an action is initiated from the remote monitoring system, a diagnostic event of the monitoring system, an alarm trigger, a mechanical trigger, and the like.

The adherent device be activated by a variety of different means including but not limited to, a physiological trigger, automatic impedance, a tab pull, battery insertion, a hall or reed switch, a breakable glass capsule, a dome switch, by light activation, pressure activation, body temperature activation, a connection between electronics associated with the sensors and the adherent device, exposure to air, by a capacitive skin sensor and the like.

The detecting system 12 can continuously, or non-continuously, monitor the patient, alerts are provided as necessary and medical intervention is provided when required. In one embodiment, the wireless communication device 16 is a wireless local area network for receiving data from the plurality of sensors.

A processor 20 is coupled to the plurality of sensors 14 and can also be a part of the wireless communication device 16. The processor 20 receives data from the plurality of sensors 14 and creates processed patient data. In one embodiment, the processor 20 is at the remote monitoring system 18. In another embodiment, the processor 20 is at the detecting system 12. The processor 20 can be integral with a monitoring unit 22 that is part of the detecting system 12 or part of the remote monitoring system 18.

The processor 20 has program instructions for evaluating values received from the sensors 14 with respect to acceptable physiological ranges for each value received by the processor 20 and determine variances. The processor 20 can receive and store a sensed measured parameter from the sensors 14, compare the sensed measured value with a predetermined target value, determine a variance, accept and store a new predetermined target value and also store a series of questions from the remote monitoring system 18.

As shown in FIG. 4, logic resources 24 are provided that take the data from the sensors 14, and/or the processed patient data from the processor 20, to predict an impending decompensation. The logic resources 24 can be at the remote monitoring system 18 or at the detecting system 12, such as in the monitoring unit 22.

In one embodiment, a memory management device 25 is provided as illustrated in FIG. 5. In various embodiments, the memory management device 25 performs one or more of data compression, prioritizing of sensing by a sensor 14, monitoring all or some of sensor data by all or a portion of the sensors 14, sensing by the sensors 14 in real time, noise blanking to provide that sensor data is not stored if a selected noise level is determined, low-power of battery caching and decimation of old sensor data.

The sensors 14 can provide a variety of different functions, including but not limited to, initiation, programming, measuring, storing, analyzing, communicating, predicting, and displaying of a physiological event of the patient. Each sensor 14 is preferably sealed, such as housed in a hermetically sealed package. In one embodiment, at least a portion of the sealed packages include a power source, a memory, logic resources and a wireless communication device. In one embodiment, the sensors 14 can include, flex circuits, thin film resistors, organic transistors and the like. The sensors 14 can include ceramics to enclose the electronics. Additionally, the sensors 14 can include drug eluting coatings, including but not limited to, an antibiotic, anti-inflammatory agent and the like.

A wide variety of different sensors 14 can be utilized, including but not limited to, bioimpedance, heart rate, heart rhythm, HRV, HRT, heart sounds, respiration rate, respiration rate variability, respiratory sounds, SpO₂, blood pressure, activity, posture, wake/sleep, orthopnea, temperature, heat flux and an accelerometer. A variety activity sensors can be utilized, including but not limited to a, ball switch, accelerometer, minute ventilation, HR, bioimpedance noise, skin temperature/heat flux, BP, muscle noise, posture, and the like.

The outputs of the sensors 14 can have multiple features to enhance physiological sensing performance. These multiple features have multiple sensing vectors that can include redundant vectors. The sensors can include current delivery electrodes and sensing electrodes. Size and shape of current delivery electrodes, and the sensing electrodes, can be optimized to maximize sensing performance. The system 10 can be configured to determine an optimal sensing configuration and electronically reposition at least a portion of a sensing vector of a sensing electrode. The multiple features enhance the system's 10 ability to determine an optimal sensing configuration and electronically reposition sensing vectors. In one embodiment, the sensors 14 can be partially masked to minimize contamination of parameters sensed by the sensors 14.

The size and shape of current delivery electrodes, for bioimpedance, and sensing electrodes can be optimized to maximize sensing performance. Additionally, the outputs of the sensors 14 can be used to calculate and monitor blended indices. Examples of the blended indices include but are not limited to, heart rate (HR) or respiratory rate (RR) response to activity, HR/RR response to posture change, HR+RR, HR/RR+bioimpedance, and/or minute ventilation/accelerometer, and the like.

The sensors 14 can be cycled in order to manage energy, and different sensors 14 can sample at different times. By way of illustration, and without limitation, instead of each sensor 14 being sampled at a physiologically relevant interval, e.g., every 30 seconds, one sensor 14 can be sampled at each interval, and sampling cycles between available sensors.

By way of illustration, and without limitation, the sensors 14 can sample 5 seconds for every minute for ECG, once a second for an accelerometer sensor, and 10 seconds for every 5 minutes for impedance.

In one embodiment, a first sensor 14 is a core sensor 14 that continuously monitors and detects, and a second sensor 14 verifies a physiological status in response to the core sensor 14 raising a flag. Additionally, some sensors 14 can be used for short term tracking, and other sensors 14 used for long term tracking.

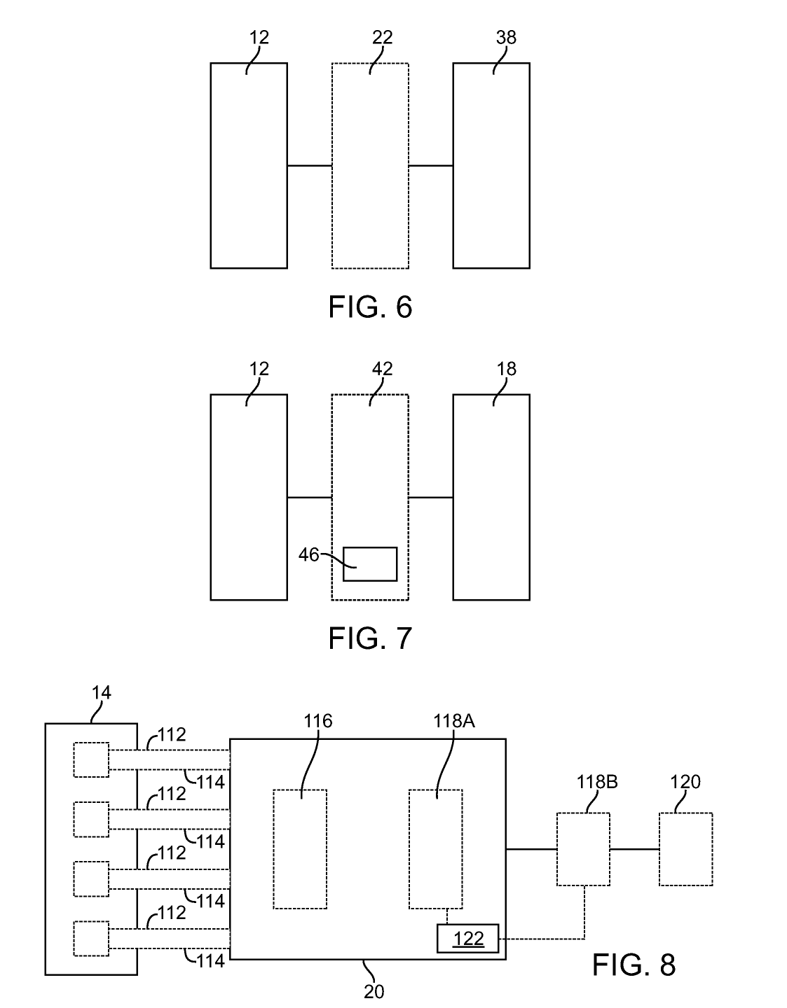

Referring to FIG. 6, an external device 38, including a medical treatment device, can be coupled to the sensors 14. The external device 38 can be coupled to a monitoring unit 22 that is part of the detecting system 12, or in direct communication with the sensors 14. A variety of different external devices 38 can be used, including but not limited to, a weight scale, blood pressure cuff, cardiac rhythm management device, a medical treatment device, medicament dispenser and the like. Suitable cardiac rhythm management devices include but are not limited to, Boston Scientific's Latitude system, Medtronic's CareLink system, St. Jude Medical's HouseCall system, and the like. Such communication may occur directly, or via an external translator unit.

The external device 38 can be coupled to an auxiliary input of the monitoring unit 22 at the detecting system 12 or to the monitoring system 22 at the remote monitoring system 18. Additionally, an automated reader can be coupled to an auxiliary input in order to allow a single monitoring unit 22 to be used by multiple patients. As previously mentioned above, the monitoring unit 22 can be at the remote monitoring system 18 and each patient can have a patient identifier (ID) including a distinct patient identifier. In addition, the ID identifier can also contain patient specific configuration parameters. The automated reader can scan the patient identifier ID and transmit the patient ID number with a patient data packet such that the main data collection station can identify the patient.

It will be appreciated that other medical treatment devices can also be used. The sensors 14 can communicate wirelessly with the external devices 38 in a variety of ways including but not limited to, a public or proprietary communication standard and the like. The sensors 14 can be configured to serve as a communication hub for multiple medical devices, coordinating sensor data and therapy delivery while transmitting and receiving data from the remote monitoring system 18.

In one embodiment, the sensors 14 coordinate data sharing between the external systems 38 allowing for sensor integration across devices. The coordination of the sensors 14 provides for new pacing, sensing, defibrillation vectors, and the like.

In one embodiment, the processor 20 is included in the monitoring unit 22 and the external device 38 is in direct communication with the monitoring unit 22.

As illustrated in FIG. 7, in another embodiment, a notification device 42 is coupled to the detecting system 12 and the remote monitoring system 18. The notification device 42 is configured to provide notification when values received from the sensors 14 are not within acceptable physiological ranges. The notification device 42 can be at the remote monitoring system 18 or at the monitoring unit 22 that is part of the detecting system 12. A variety of notification devices 42 can be utilized, including but not limited to, a visible patient indicator, an audible alarm, an emergency medical service notification, a call center alert, direct medical provider notification and the like. The notification device 42 provides notification to a variety of different entities, including but not limited to, the patient, a caregiver, the remote monitoring system, a spouse, a family member, a medical provider, from one device to another device such as the external device 38, and the like.

Notification can be according to a preset hierarchy. By way of illustration, and without limitation, the preset hierarchy can be, patient notification first and medical provider second, patient notification second and medical provider first, and the like. Upon receipt of a notification, a medical provider, the remote monitoring system 18, or a medical treatment device can trigger a high-rate sampling of physiological parameters for alert verification.

The system 10 can also include an alarm 46, that can be coupled to the notification device 42, for generating a human perceptible signal when values received from the sensors 14 are not within acceptable physiological ranges. The alarm 46 can trigger an event to render medical assistance to the patient, provide notification as set forth above, continue to monitor, wait and see, and the like.

When the values received from the sensors 14 are not within acceptable physiological ranges the notification is with the at least one of, the patient, a spouse, a family member, a caregiver, a medical provider and from one device to another device, to allow for therapeutic intervention to prevent decompensation, and the like.

In another embodiment, the sensors 14 can switch between different modes, wherein the modes are selected from at least one of, a stand alone mode with communication directly with the remote monitoring system 18, communication with implanted devices, communication with a single implanted device, coordination between different devices (external systems) coupled to the plurality of sensors and different device communication protocols.

By way of illustration, and without limitation, the patient can be a congestive heart failure patient. Heart failure status is determined by a weighted combination change in sensor outputs and be determined by a number of different means, including but not limited to, (i) when a rate of change of at least two sensor outputs is an abrupt change in the sensor outputs as compared to a change in the sensor outputs over a longer period of time, (ii) by a tiered combination of at least a first and a second sensor output, with the first sensor output indicating a problem that is then verified by at least a second sensor output, (iii) by a variance from a baseline value of sensor outputs, and the like. The baseline values can be defined in a look up table.

In another embodiment, heart failure status is determined using three or more sensors by at least one of, (i) when the first sensor output is at a value that is sufficiently different from a baseline value, and at least one of the second and third sensor outputs is at a value also sufficiently different from a baseline value to indicate heart failure status, (ii) by time weighting the outputs of the first, second and third sensors, and the time weighting indicates a recent event that is indicative of the heart failure status, and the like.

In one embodiment, the wireless communication device 16 can include a, modem, a controller to control data supplied by the sensors 14, serial interface, LAN or equivalent network connection and a wireless transmitter. Additionally, the wireless communication device 16 can include a receiver and a transmitter for receiving data indicating the values of the physiological event detected by the plurality of sensors, and for communicating the data to the remote monitoring system 18. Further, the wireless communication device 16 can have data storage for recording the data received from the sensors 14 and an access device for enabling access to information recording in the data storage from the remote monitoring system 18.

In various embodiments, the remote monitoring system 18 can include a receiver, a transmitter and a display for displaying data representative of values of the one physiological event detected by the sensors 14. The remote monitoring system can also include a, data storage mechanism that has acceptable ranges for physiological values stored therein, a comparator for comparing the data received from the monitoring system 12 with the acceptable ranges stored in the data storage device and a portable computer. The remote monitoring system 18 can be a portable unit with a display screen and a data entry device for communicating with the wireless communication device 16.

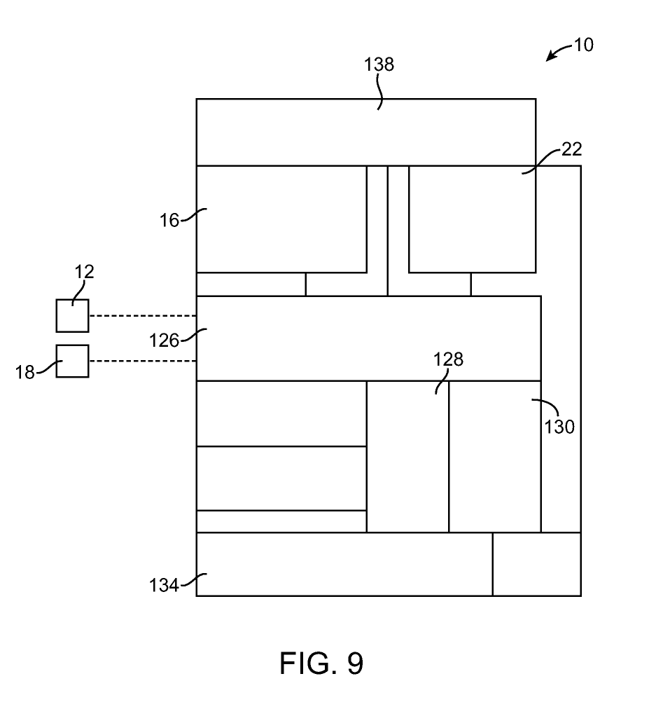

Referring now to FIG. 8, for each sensor 14, a sensor lead 112 and 114 conveys signals from the sensor 14 to the monitoring unit 22 at the detecting system 12, or through the wireless communication device 16 to the remote monitoring system 18. In one embodiment, each signal from a sensor 14 is first passed through a low-pass filter 116, at the detecting system 12 or at the remote monitoring system 18, to smooth the signal and reduce noise. The signal is then transmitted to an analog-to-digital converter 118A, which transforms the signals into a stream of digital data values, that can be stored in a digital memory 118B. From the digital memory 118B, data values are transmitted to a data bus 120, along which they are transmitted to other components of the circuitry to be processed and archived. From the data bus 120, the digital data can be stored in a non-volatile data archive memory. The digital data can be transferred via the data bus 120 to the processor 20, which processes the data based in part on algorithms and other data stored in a non-volatile program memory.

The detecting system 12 can also include a power management module 122 configured to power down certain components of the system, including but not limited to, the analog-to-digital converters 118A, digital memories 118B and the non-volatile data archive memory and the like, between times when these components are in use. This helps to conserve battery power and thereby extend the useful life. Other circuitry and signaling modes may be devised by one skilled in the art.

As can be seen in FIG. 9, a control unit 126 is included at the detecting system 12, the remote monitoring system 18 or at both locations.

In one embodiment, the control unit 126 can be a 486 microprocessor. The control unit 126 can be coupled to the sensors 14 directly at the detecting system 12, indirectly at the detecting system 12 or indirectly at the remote monitoring system 18. Additionally the control unit 126 can be coupled to a, blood pressure monitor, cardiac rhythm management device, scale, a device that dispenses medication that can indicate the medication has been dispensed.

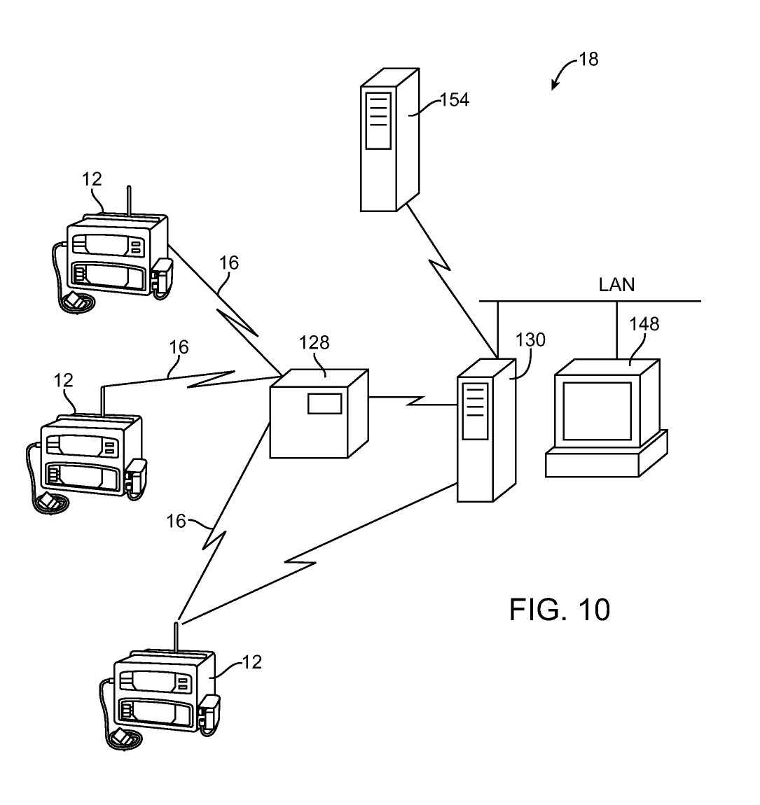

The control unit 126 can be powered by AC inputs which are coupled to internal AC/DC converters 134 that generate multiple-DC voltage levels. After the control unit 126 has collected the patient data from the sensors 14, the control unit 126 encodes the recorded patient data and transmits the patient data through the wireless communication device 16 to transmit the encoded patient data to a wireless network storage unit 128 at the remote monitoring system 18 as shown in FIG. 10. In another embodiment, wireless communication device 16 transmits the patient data from the sensors 14 to the control unit 126 when it is at the remote monitoring system 18.

Every time the control unit 126 plans to transmit patient data to a main data collection station 130, located at the remote monitoring system 18, the control unit 126 attempts to establish a communication link. The communication link can be wireless, wired, or a combination of wireless and wired for redundancy, e.g., the wired link checks to see if a wireless communication can be established. If the wireless communication link 16 is available, the control unit 126 transmits the encoded patient data through the wireless communication device 16. However, if the wireless communication device 16 is not available for any reason, the control unit 126 waits and tries again until a link is established.

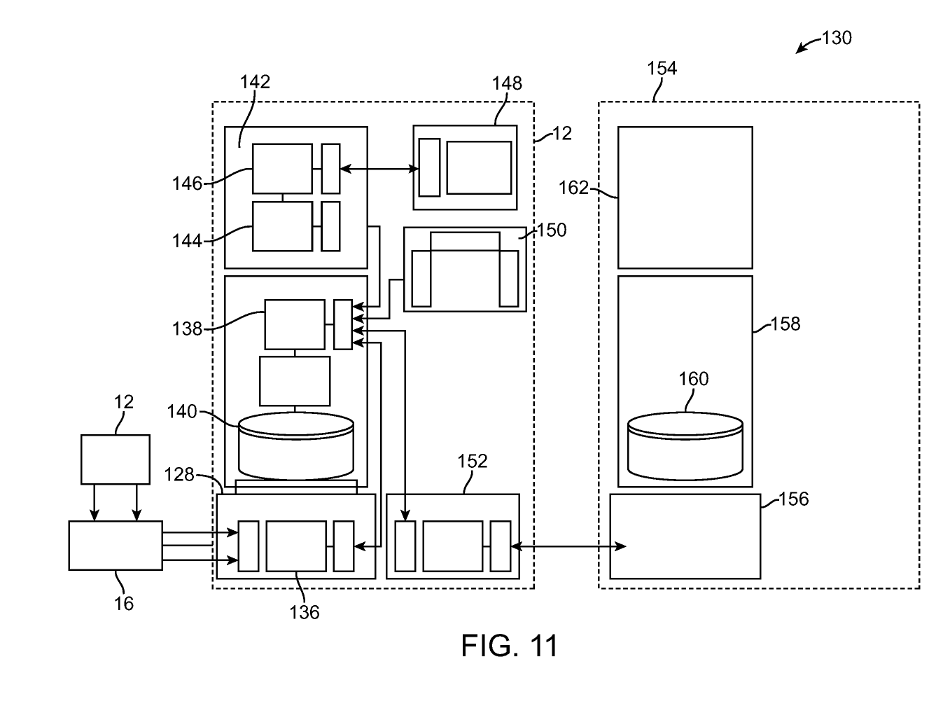

Referring now to FIG. 11, one embodiment of an internal structure of a main data collection station 130 at the remote monitoring system 18, is illustrated. The patient data can be transmitted by the remote monitoring system 18 by either the wireless communication device 16 or conventional modem to the wireless network storage unit 128. After receiving the patient data, the wireless network storage unit 128 can be accessed by the main data collection station 130. The main data collection station 130 allows the remote monitoring system 18 to monitor the patient data of numerous patients from a centralized location without requiring the patient or a medical provider to physically interact with each other.

The main data collection station 130 can include a communications server 136 that communicates with the wireless network storage unit 128. The wireless network storage unit 128 can be a centralized computer server that includes a unique, password protected mailbox assigned to and accessible by the main data collection station 130. The main data collection station 130 contacts the wireless network storage unit 128 and downloads the patient data stored in a mailbox assigned to the main data collection station 130.

Once the communications server 136 has formed a link with the wireless network storage unit 128, and has downloaded the patient data, the patient data can be transferred to a database server 138. The database server 138 includes a patient database 140 that records and stores the patient data of the patients based upon identification included in the data packets sent by each of the monitoring units 22. For example, each data packet can include an identifier.

Each data packet transferred from the remote monitoring system 18 to the main data collection station 130 does not have to include any patient identifiable information. Instead, the data packet can include the serial number assigned to the specific detecting system 12. The serial number associated with the detecting system 12 can then be correlated to a specific patient by using information stored on the patient database 138. In this manner, the data packets transferred through the wireless network storage unit 128 do not include any patient-specific identification. Therefore, if the data packets are intercepted or improperly routed, patient confidentiality cannot be breached.

The database server 138 can be accessible by an application server 142. The application server 142 can include a data adapter 144 that formats the patient data information into a form that can be viewed over a conventional web-based connection. The transformed data from the data adapter 144 can be accessible by propriety application software through a web server 146 such that the data can be viewed by a workstation 148. The workstation 148 can be a conventional personal computer that can access the patient data using proprietary software applications through, for example, HTTP protocol, and the like.

The main data collection station further can include an escalation server 150 that communicates with the database server 138. The escalation server 150 monitors the patient data packets that are received by the database server 138 from the monitoring unit 22. Specifically, the escalation server 150 can periodically poll the database server 138 for unacknowledged patient data packets. The patient data packets are sent to the remote monitoring system 18 where the processing of patient data occurs. The remote monitoring system 18 communicates with a medical provider in the event that an alert is required. The escalation server 150 can be programmed to automatically deliver alerts to a specific medical provider if an alarm message has not been acknowledged within a selected time period after receipt of the data packet.

The escalation server 150 can be configured to generate the notification message to different people by different modes of communication after different delay periods and during different time periods.

The main data collection station 130 can include a batch server 152 connected to the database server 138. The batch server 152 allows an administration server 154 to have access to the patient data stored in the patient database 140. The administration server 154 allows for centralized management of patient information and patient classifications.

The administration server 154 can include a batch server 156 that communicates with the batch server 152 and provides the downloaded data to a data warehouse server 158. The data warehouse server 158 can include a large database 160 that records and stores the patient data.

The administration server 154 can further include an application server 162 and a maintenance workstation 164 that allow personnel from an administrator to access and monitor the data stored in the database 160.

The data packet utilized in the transmission of the patient data can be a variable length ASCII character packet, or any generic data formats, in which the various patient data measurements are placed in a specific sequence with the specific readings separated by commas. The control unit 126 can convert the readings from each sensor 14 into a standardized sequence that forms part of the patient data packet. In this manner, the control unit 126 can be programmed to convert the patient data readings from the sensors 14 into a standardized data packet that can be interpreted and displayed by the main data collection station 130 at the remote monitoring system 18.

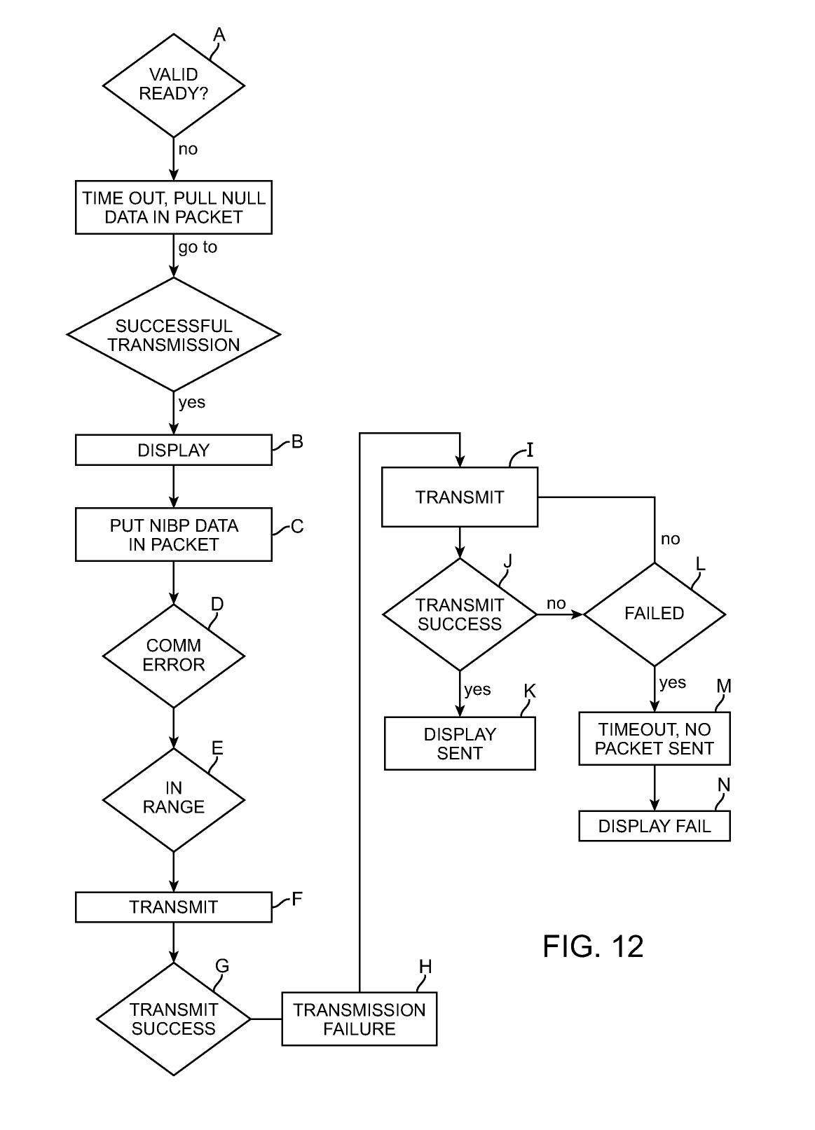

Referring now to the flow chart of FIG. 12, if an external device 38 fails to generate a valid reading, as illustrated in step A, the control unit 126 fills the portion of the patient data packet associated with the external device 38 with a null indicator. The null indicator can be the lack of any characters between commas in the patient data packet. The lack of characters in the patient data packet can indicate that the patient was not available for the patient data recording. The null indicator in the patient data packet can be interpreted by the main data collection station 130 at the remote monitoring system 18 as a failed attempt to record the patient data due to the unavailability of the patient, a malfunction in one or more of the sensors 14, or a malfunction in one of the external devices 38. The null indicator received by the main data collection station 130 can indicate that the transmission from the detecting system 12 to the remote monitoring system 18 was successful. In one embodiment, the integrity of the data packet received by the main data collection station 130 can be determined using a cyclic redundancy code, CRC-16, check sum algorithm. The check sum algorithm can be applied to the data when the message can be sent and then again to the received message.

After the patient data measurements are complete, the control unit 126 displays the sensor data, including but not limited to blood pressure cuff data and the like, as illustrated by step B. In addition to displaying this data, the patient data can be placed in the patient data packet, as illustrated in step C.

As previously described, the system 10 can take additional measurements utilizing one or more auxiliary or external devices 38 such as those mentioned previously. Since the patient data packet has a variable length, the auxiliary device patient information can be added to the patient data packet being compiled by the remote monitoring unit 22 during patient data acquisition period being described. Data from the external devices 38 is transmitted by the wireless communication device 16 to the remote monitoring system 18 and can be included in the patient data packet.

If the remote monitoring system 18 can be set in either the auto mode or the wireless only mode, the remote monitoring unit 22 can first determine if there can be an internal communication error, as illustrated in step D.

A no communication error can be noted as illustrated in step E. If a communication error is noted the control unit 126 can proceed to wireless communication device 16 or to a conventional modem transmission sequence, as will be described below. However, if the communication device is working the control unit 126 can transmit the patient data information over the wireless network 16, as illustrated in step F. After the communication device has transmitted the data packet, the control unit 126 determines whether the transmission was successful, as illustrated in step G. If the transmission has been unsuccessful only once, the control unit 126 retries the transmission. However, if the communication device has failed twice, as illustrated in step H, the control unit 126 proceeds to the conventional modem process if the remote monitoring unit 22 was configured in an auto mode.

When the control unit 126 is at the detecting system 12, and the control unit 126 transmits the patient data over the wireless communication device 16, as illustrated in step I, if the transmission has been successful, the display of the remote monitoring unit 22 can display a successful message, as illustrated in step J. However, if the control unit 126 determines in step K that the communication of patient data has failed, the control unit 126 repeats the transmission until the control unit 126 either successfully completes the transmission or determines that the transmission has failed a selected number of times, as illustrated in step L. The control unit 126 can time out the and a failure message can be displayed, as illustrated in steps M and N. Once the transmission sequence has either failed or successfully transmitted the data to the main data collection station, the control unit 126 returns to a start program.

As discussed previously, the patient data packets are first sent and stored in the wireless network storage unit 128. From there, the patient data packets are downloaded into the main data collection station 130. The main data collection station 130 decodes the encoded patient data packets and records the patient data in the patient database 140. The patient database 140 can be divided into individual storage locations for each patient such that the main data collection station 130 can store and compile patient data information from a plurality of individual patients.

A report on the patient's status can be accessed by a medical provider through a medical provider workstation that is coupled to the remote monitoring system 18. Unauthorized access to the patient database can be prevented by individual medical provider usernames and passwords to provide additional security for the patient's recorded patient data.

The main data collection station 130 and the series of work stations 148 allow the remote monitoring system 18 to monitor the daily patient data measurements taken by a plurality of patients reporting patient data to the single main data collection station 130. The main data collection station 130 can be configured to display multiple patients on the display of the workstations 148. The internal programming for the main data collection station 130 can operate such that the patients are placed in a sequential top-to-bottom order based upon whether or not the patient can be generating an alarm signal for one of the patient data being monitored. For example, if one of the patients monitored by monitoring system 130 has a blood pressure exceeding a predetermined maximum amount, this patient can be moved toward the top of the list of patients and the patient's name and/or patient data can be highlighted such that the medical personnel can quickly identify those patients who may be in need of medical assistance.

By way of illustration, and without limitation, the following paragraphs is a representative order ranking method for determining the order which the monitored patients are displayed:

Alarm Display Order Patient Status Patients are then sorted 1 Medical Alarm Most alarms violated to least alarms violated, then oldest to newest 2 Missing Data Alarm Oldest to newest 3 Late Oldest to newest 4 Reviewed Medical Alarms Oldest to newest 5 Reviewed Missing Data Oldest to newest Alarms 6 Reviewed Null Oldest to newest 7 NDR Oldest to newest 8 Reviewed NDR Oldest to newest.

As listed in the above, the order of patients listed on the display can be ranked based upon the seriousness and number of alarms that are registered based upon the latest patient data information. For example, if the blood pressure of a single patient exceeds the tolerance level and the patient's heart rate also exceeds the maximum level, this patient will be placed above a patient who only has one alarm condition. In this manner, the medical provider can quickly determine which patient most urgently needs medical attention by simply identifying the patient's name at the top of the patient list. The order which the patients are displayed can be configurable by the remote monitoring system 18 depending on various preferences.

As discussed previously, the escalation server 150 automatically generates a notification message to a specified medical provider for unacknowledged data packets based on user specified parameters.

In addition to displaying the current patient data for the numerous patients being monitored, the software of the main data collection station 130 allows the medical provider to trend the patient data over a number of prior measurements in order to monitor the progress of a particular patient. In addition, the software allows the medical provider to determine whether or not a patient has been successful in recording their patient data as well as monitor the questions being asked by the remote monitoring unit 22.

As previously mentioned, the system 10 uses an intelligent combination of sensors to enhance detection and prediction capabilities. Electrocardiogram circuitry can be coupled to the sensors 14, or electrodes, to measure an electrocardiogram signal of the patient. An accelerometer can be mechanically coupled, for example adhered or affixed, to the sensors 14, adherent patch and the like, to generate an accelerometer signal in response to at least one of an activity or a position of the patient. The accelerometer signals improve patient diagnosis, and can be especially useful when used with other signals, such as electrocardiogram signals and impedance signals, including but not limited to, hydration respiration, and the like. Mechanically coupling the accelerometer to the sensors 14, electrodes, for measuring impedance, hydration and the like can improve the quality and/or usefulness of the impedance and/or electrocardiogram signals. By way of illustration, and without limitation, mechanical coupling of the accelerometer to the sensors 14, electrodes, and to the skin of the patient can improve the reliability, quality and/or accuracy of the accelerometer measurements, as the sensor 14, electrode, signals can indicate the quality of mechanical coupling of the patch to the patient so as to indicate that the device is connected to the patient and that the accelerometer signals are valid. Other examples of sensor interaction include but are not limited to, (i) orthopnea measurement where the breathing rate is correlated with posture during sleep, and detection of orthopnea, (ii) a blended activity sensor using the respiratory rate to exclude high activity levels caused by vibration (e.g., driving on a bumpy road) rather than exercise or extreme physical activity, (iii) sharing common power, logic and memory for sensors, electrodes, and the like.

While the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention.

What is claimed is:

1. A system for physiological monitoring and prediction, comprising:
a detecting system including:
an adherent device configured to be coupled to a patient, the adherent device including a plurality of sensors that provide an indication of at least one physiological event of a patient,
a wireless communication device coupled to the plurality of sensors and configured to transfer patient data from the plurality of sensors and one or more medical treatment devices to a remote monitoring system; and
one or more medical treatment devices coupled to the wireless communication device to communicate wirelessly with the plurality of sensors, wherein at least one medical treatment device is an implanted device;
a remote monitoring system coupled to the wireless communication device, wherein the remote monitoring system uses data from the sensors and the one or more medical treatment devices to determine heart failure status and predict impending decompensation of the patient.

2. The system of claim 1, wherein the plurality of sensors serve as a communication hub for the one or more medical devices.

3. The system of claim 1, wherein each of a sensor is selected from at least one of, a bioimpedance sensor, a heart rate sensor, a heart rhythm sensor, a HRV sensor, HRT sensor, a heart sounds sensor, a respiration rate sensor, a respiration rate variability sensor, a respiratory sounds sensor, a SpO₂ sensor, a blood pressure sensor, an activity sensor, a posture sensor, a wake/sleep sensor, an orthopnea sensor, a temperature sensor, a heat flux sensor and an accelerometer.

4. The system of claim 1, wherein the medical treatment device is a cardiac rhythm management device.

5. The system of claim 1, wherein the communication includes coordinating sensor data and therapy delivery while transmitting and receiving data from the remote monitoring system.

6. The system of claim 1, wherein the plurality of sensors is configured to communicate with external systems.

7. The system of claim 6, wherein the external systems are selected from at least one of, a weight scale, blood pressure cuff, cardiac rhythm management device, a medical treatment device and a medicament dispenser.

8. The system of claim 1, wherein the plurality of sensors is configured to coordinate data sharing between devices allowing for sensor integration across the devices.

9. The system of claim 8, wherein the devices are selected from at least one of, a weight scale, blood pressure cuff, cardiac rhythm management device, a medical treatment device and a medicament dispenser.

10. The system of claim 8, wherein coordination of sensors of the plurality of sensors provides for new pacing, sensing, and defibrillation vectors.

11. The system of claim 1, wherein the plurality of sensors is configured to switch between different modes.

12. The system of claim 11, wherein the modes are selected from at least one of, a stand alone mode with communication directly with a remote monitoring system, communication directly with any one of the one or more medical devices, coordination between the one or more medical devices coupled to the plurality of sensors and different device communication protocols.

13. The system of claim 1, wherein the plurality of sensors is configured to deactivate selected sensors to reduce redundancy.

14. An adherent multi-sensor device comprising:
a plurality of sensors that provide an indication of at least one physiological event of a patient,
a processor couple to the plurality of sensors to receive data from the plurality of sensors and creates processed patient data; and
a wireless communication device coupled to the processor and configured to receive patient data from one or more medical treatment devices wherein at least one medical treatment device is an implanted device, the adherent multi-sensor device serving as a communication hub for the one or more medical devices.

15. The device of claim 14, wherein the wireless communication device communicates data from the processor, sensors and/or the one or more medical treatment devices to a remote monitoring system.

16. The device of claim 14, wherein the wireless communication device is configured to receive data from a remote monitoring system and coordinate therapy delivery for the one or more medical devices.

17. The device of claim 14, wherein the plurality of sensors is configured to coordinate data sharing between the one or more medical devices allowing for sensor integration across the one or more medical devices.

18. The device of claim 14, wherein each of a sensor is selected from at least one of, a bioimpedance sensor, a heart rate sensor, a heart rhythm sensor, a HRV sensor, HRT sensor, a heart sounds sensor, a respiration rate sensor, a respiration rate variability sensor, a respiratory sounds sensor, a SpO₂ sensor, a blood pressure sensor, an activity sensor, a posture sensor, a wake/sleep sensor, an orthopnea sensor, a temperature sensor, a heat flux sensor and an accelerometer.

19. The device of claim 14, wherein the plurality of sensors is configured to switch between different modes selected from at least one of, a stand alone mode with communication directly with a remote monitoring system, communication directly with any one of the one or more medical devices, coordination between the one or more medical devices coupled to the plurality of sensors and different device communication protocols.

20. The device of claim 14, wherein the plurality of sensors is configured to deactivate selected sensors to reduce redundancy.
